(19) United States
(12) Patent Application Publication
(10) Pub. No.: US 2003/0204244 A1
(43) Pub. Date: Oct. 30, 2003

(54) Aneurysm exclusion stent

(76) Inventor: Mark L. Stiger, Windsor, CA (US)

Correspondence Address: MEDTRONIC AVE, INC., 3576 UNOCAL PLACE, SANTA ROSA, CA, 95403, US

(21) Appl. No.: 10/133,131

(22) Filed: Apr. 26, 2002

(51) Int. Cl. A61F 2/06

(52) U.S. Cl. 623/1.16

(57) ABSTRACT

An exclusion stent includes a proximal anchor and distal anchor for anchoring the exclusion stent within a parent vessel. A center mesh is connected to and extends between the proximal anchor and the distal anchor. During use, the exclusion stent is deployed such that the center mesh extends across an aneurysm. The center mesh restricts blood flow to the aneurysm. However, the center mesh does not completely block blood flow, thus insuring blood flow to perforators of the parent vessel.

### BACKGROUND OF THE INVENTION

1. Field Of The Invention

The present invention relates to an intra-vascular device and method. More particularly, the present invention relates to a stent for treatment of intra-cranial aneurysms.

2. Description Of The Related Art

Several procedures and methods have been established for the exclusion of intra-vascular aneurysm. The coil precipitated the formation of a thrombus, or clot, within the aneurysm. The thrombus partially or completely occluded the aneurysm. In this manner, blood from the parent artery (or vessel) was prevented from flowing into and circulating within the aneurysm.

This method posed the risk of the coil or ensuing thrombus migrating from the aneurysm to the parent artery and causing a thrombo-embolic stroke. This method was also limited to the treatment of saccular aneurysms with small necks in order to ensure that the coil remained within the aneurysm.

Another method of treating saccular aneurysms was to employ a stent. The stent was anchored on either side of the aneurysm. The stent was anchored such that a diverter of the stent was positioned overlying the neck of the aneurysm. To obtain the proper radial orientation of the diverter, the operating surgeon often had to repeatedly rotate, insert and withdraw the stent prior to permanent deployment. This increased the time of the procedure and was accompanied by the inherent risk that the stent would be deployed without the proper radial orientation.

Another method of treating both saccular and fistula aneurysms was to deploy a graft. The graft was anchored on either side of the aneurysm and extended across the aneurysm. Since the graft extended across the aneurysm, the flow of blood to the aneurysm was blocked thus relieving the pressure on the aneurysm.

Although potentially successful in such applications as abdominal aortic aneurysms, grafts were not well-suited for intra-cranial aneurysm applications. As is well known, any device placed in the parent artery of an intra-cranial aneurysm runs the risk of occluding perforating side branches, sometimes called perforators. Grafts could easily partially or completely block the flow of blood to one or more such perforators, thereby causing clinically significant ischemic strokes. Improvements in treating intra-cranial aneurysms are continually being sought to achieve improved treatment of such conditions.

### SUMMARY OF THE INVENTION

In accordance with one embodiment of the present invention, an exclusion stent includes a proximal anchor and distal anchor for anchoring the exclusion stent within a parent vessel. A center mesh is connected to and extends between the proximal anchor and the distal anchor. The center mesh is semi permeable to blood.

During use, the proximal anchor and the distal anchor are anchored to the parent vessel on either side of an aneurysm such that the center mesh extends across the aneurysm. In this manner, the center mesh restricts blood flow to the aneurysm. However, since the center mesh is semi permeable, the center mesh only restricts blood flow, i.e., does not completely block blood flow, thus insuring blood flow to perforators of the parent vessel.

The present invention is best understood by reference to the following detailed description when read in conjunction with the accompanying drawings.

### BRIEF DESCRIPTION OF THE DRAWINGS

FIG. 1 is a perspective view of an exclusion stent in accordance with an embodiment of the present invention;

FIG. 2 is a plan view of an exclusion stent pattern in accordance with one embodiment of the present invention;

FIG. 3 is a plan view of an exclusion stent pattern in accordance with another embodiment of the present invention;

FIG. 4 is a cross-sectional view of a saccular aneurysm having a neck to a parent artery or vessel of a patient in which the exclusion stent of FIG. 2 is deployed;

FIG. 5 is a cross-sectional view of a fistula aneurysm in a parent artery or vessel of a patient in which the exclusion stent of FIG. 2 is deployed;

FIG. 6 is a schematic view of a delivery system for deploying the exclusion stent of FIG. 2;

FIG. 7 is a close up view showing region VII of the exclusion stent of FIG. 2;

FIG. 8 shows two approximations of cross sectional views of a proximal anchor along the line VIII-VIII of the exclusion stent of FIG. 2, an expanded state stent cross section is superimposed over a contracted state stent cross section; and

FIG. 9 shows two approximations of cross sectional views of a center mesh along the line IX-IX of the exclusion stent of FIG. 2 an expanded state stent cross section is superimposed over a contracted state stent cross section.

Common reference numerals are used throughout the drawings and detailed description to indicate like elements.

### DETAILED DESCRIPTION

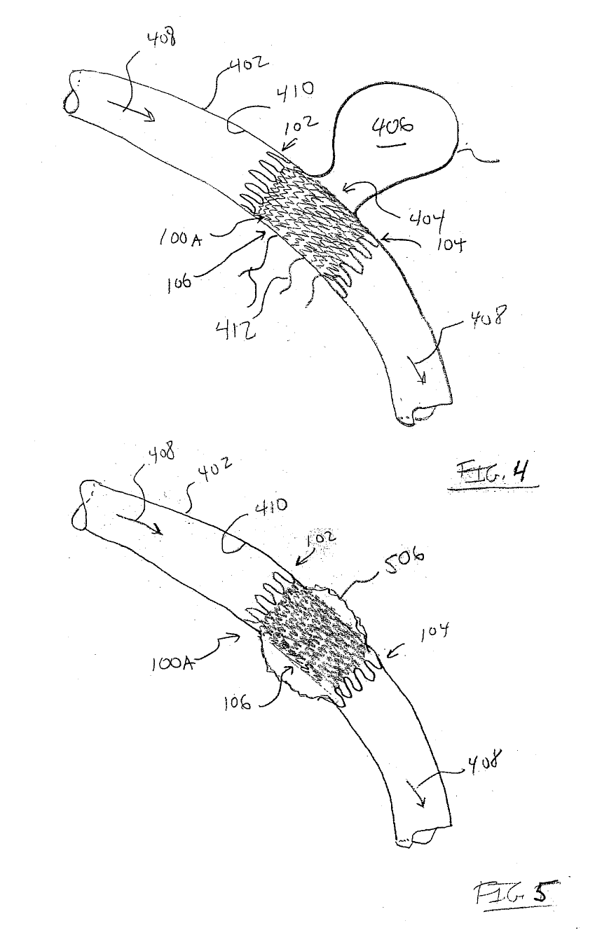

An exclusion stent 100A (FIG. 4) includes a proximal anchor stent ring 102 and a distal anchor stent ring 104 anchoring exclusion stent portion 100A within a parent vessel 402. The anchoring exclusion portion 100A is a center mesh 106 semi permeable to blood.

While semi permeable to blood has a general meaning, in this application, its definition is restricted to center mesh sections where the material (e.g., usually metal) to artery ratios at the design diameter of the stent is in the range of 18% to 50% (or more narrowly 20% to 45%). That is, given a vessel of a particular diameter the assumption is that its shape is a symmetrical butt end cylinder, so the denominator of the ratio is the cylinder wall area for a unit distance, the numerator is the material coverage of the cylindrical wall when expanded to the design diameter of the stent (generally precisely set by the balloon expansion to within 0.005 of an inch) for a full cycle of an axially repeating pattern. Thus the material artery ratio is calculable. The center mesh 106 extends across the opening to an aneurysm 406 and restricts blood flow to aneurysm 406. However, since the center mesh 106 is semi permeable, the center mesh 106 only restricts blood flow, i.e., does not completely block blood flow, thus insuring blood flow to perforators 412 (perforators are very small blood vessels (approx 0.5 mm in dia. which emanate from the side of small branch vessels 3 to 4 mm dia) of parent vessel 402 (small branch vessel). Further being semi-permeable to blood in a definition according to the invention further includes the proviso that the diameter of the components of the center mesh portion be generally small so that a crossing profile of two components (wires or braid components) when crossing in front of the perforator opening having an approximate diameter of 0.5 mm, do not theoretically obstruct more than 50% of the area of the perforator opening when a plan view of the crossing profile is projected to a planar projection of the area of a 0.5 mm diameter perforator opening facing the stent. Thus the diameter (more precisely—projected area) of individual elements of the center mesh section which may cross such a perforator opening cannot be very large (e.g., wire diameters of 0.000785 and 0.001 inches might be used). Thus 50% or more of the perforator opening is available for flow and since that flow is continuous, coagulation and clotting and resulting stoppage of blood flow is unlikely to occur. So unlike prior art exclusion stents when there was an effort to place a small patch right over the aneurysmal opening requiring precise rotational orientation for proper placement, a configuration according to the present invention is the same all around, but nevertheless allows blood flow to perforators which draw blood through the center mesh, while simultaneously creating a quiescent zone (nearly if not completely eliminating the blood pressure pulsatile effect) behind the center mesh in which thrombus can form and healing or stabilization of the aneurysm can take place. Another design consideration which is important to doctors implanting these types of stents is “crossing profile,” i.e., the diameter of the stent (stent mounted on the delivery system) to cross a narrow region in an arterial passage. Stent designs with crossing profiles in excess of 0.060 inches are not likely to be acceptable to such users.

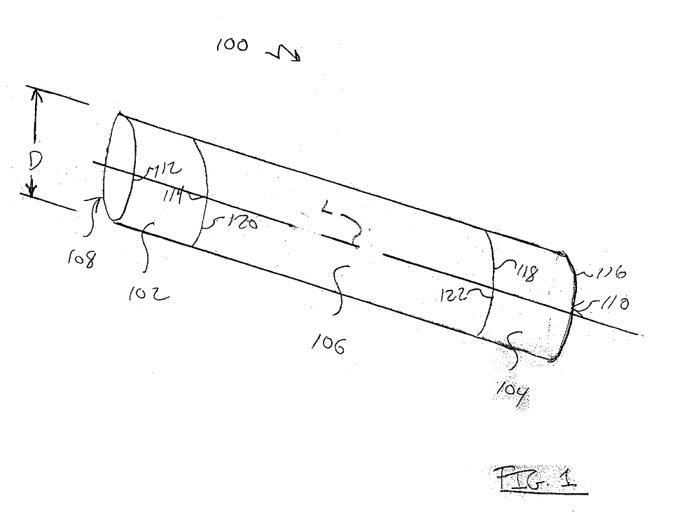

More particularly, FIG. 1 is a perspective view of an exclusion stent 100 in accordance with one embodiment of the present invention. Exclusion stent 100 is cylindrical and includes a proximal, e.g., first, anchor 102, a distal, e.g., second, anchor 104 and center mesh 106. Further, exclusion stent 100 has a longitudinal axis L.

More particularly, proximal anchor 102 is cylindrical, distal anchor 104 is cylindrical, and center mesh 106 is cylindrical. In one embodiment, proximal anchor portion (ring) 102, distal anchor ring (portion) 104, and center mesh 106 have approximately the same diameter D such that exclusion stent 100 is a uniform cylinder. However, in alternative embodiments, the center mesh section 106 has a diameter greater than or less than the diameter of proximal anchor portion 102 and/or distal anchor portion 104.

Further, although exclusion stent 100 is described above as being cylindrical, in an alternative embodiment, exclusion stent 100 can be eccentric, i.e., non cylindrical.

Exclusion stent 100 includes a proximal, e.g., first, end 108 and a distal, e.g., second, end 110. Proximal anchor 102 is located at proximal end 108 and distal anchor 104 is located at distal end 110. Center mesh 106 extends between proximal anchor 102 and distal anchor 104.

More particularly, an outer, e.g., first, edge 112 of proximal anchor 102 is at proximal end 108. Proximal anchor 102 includes an inner, e.g., second, edge 114. Similarly, an outer, e.g., first, edge 116 of distal anchor 104 is at distal end 110. Distal anchor 104 includes an inner, e.g., second, edge 118. Center mesh 106 includes a proximal, e.g., first, edge 120 connected to inner edge 114 of proximal anchor 102. Center mesh 106 further includes a distal, e.g., second, edge 122 connected to inner edge 118 of distal anchor 104.

As described in greater detail below with reference to FIGS. 4 and 5, expansion of proximal anchor portion 102 and distal anchor portion 104 causes the exclusion stent 100 to securely engage (or anchor) within the parent vessel. Center mesh 106 is semi permeable to blood. Center mesh 106 extends across the aneurysm and restricts blood flow to the aneurysm. However, since center mesh 106 is semi permeable, center mesh 106 only restricts blood flow, i.e., does not completely block blood flow, thus insuring blood flow to perforators of the parent vessel.

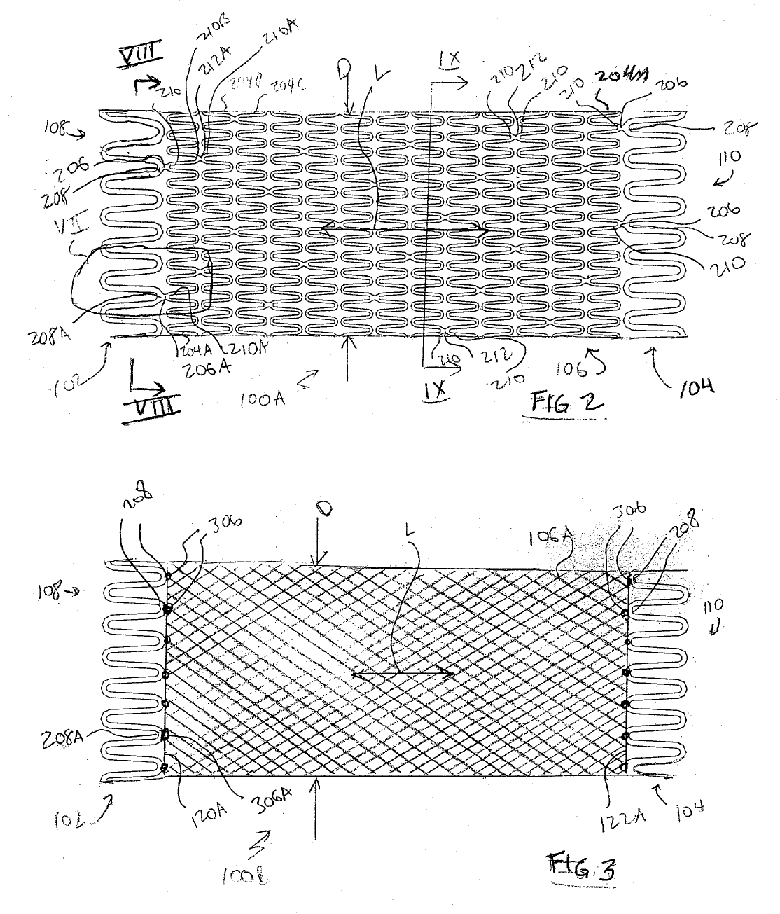

FIG. 2 is a side plan view of an exclusion stent 100A in accordance with another embodiment of the present invention. As shown in FIG. 2, proximal anchor 102 is a serpentine ring. More particularly, proximal anchor 102 has a pattern, and this pattern is sometimes called serpentine or an alternating repeating pattern.

Stated another way, proximal anchor 102 has the pattern of a sine wave, and is sometimes said to have a sinusoidal pattern. More particularly, the sine wave extends around a cylindrical surface having longitudinal axis L. Distal anchor 104 is, apart from being at the other end of the central port, identical to proximal anchor 102 and as such is not discussed further.

Center mesh 106 includes a plurality a serpentine rings 204A-204M (not all labelled in FIG. 2) connected together. In this embodiment, center mesh 106 includes thirteen serpentine rings 204A-204M. However, in alternative embodiments, center mesh 106 includes more or less than thirteen serpentine rings 204A-204M. The center mesh 106 is sometimes called a serpentine braid.

In this embodiment, each serpentine ring 204A-204M has a diameter D equal to the diameter of proximal anchor 102. However, in alternative embodiments (not shown, as such construction would be easily understood to persons skilled in the art), each serpentine ring 204A-204M has a diameter greater than or less than the diameter of proximal anchor 102.

As shown in FIG. 2, each serpentine ring 204A-204M has a pattern, and this pattern is sometimes called serpentine or an alternating repeating pattern. Stated another way, each serpentine ring 204A-204M has the pattern of a sine wave, and is sometimes said to have a sinusoidal pattern. More particularly, the sine wave extends around a cylindrical surface having longitudinal axis L.

As shown in FIG. 2, each serpentine ring 204A-204M has the same pattern as proximal anchor 102, but the pattern is smaller. More particular, the pitch (distance between adjacent crowns) of the sine wave pattern of each serpentine ring 204A-204M is less than the pitch of the sine wave pattern of proximal anchor 102. Further, the height, sometimes called amplitude, of the sine wave pattern of each serpentine ring 204A-204M is less than the height of the sine wave pattern of proximal anchor 102.

Proximal anchor 102 is connected to center mesh 106, and, more particularly, to a first serpentine ring 204A of the plurality of serpentine rings 204 by bridges 206, sometimes called outer or first bridges. Bridges 206 extend between selected peaks 208 of the sine wave pattern of proximal anchor 102 and selected valleys 210 of the sine wave pattern of serpentine ring 204A of center mesh 106. peaks and valleys 208 and 210 are sometimes called minima/maxima of the sine wave patterns of proximal anchor 102 and center mesh 106, respectively.

To illustrate, (as shown in FIG. 7 ???) a first bridge 206A of the plurality of bridges 206 extends between a first peak 208A of the plurality of peaks 208 of the sine wave pattern of proximal anchor 102 and a first valley 210A of the plurality of valleys 210 of the sine wave pattern of serpentine ring 204A of center mesh 106.

Distal anchor 104 is connected to center mesh 106, and, more particularly, to a last serpentine ring 204M of the plurality of serpentine rings 204 by bridges 206 in a similar manner except that peaks of the serpentine ring 204M are connected to valleys of the distal anchor and so is not discussed further.

Serpentine rings 204A-204M, collectively referred to as serpentine rings 204, are connected to one another by bridges 212, sometimes called inter or second bridges. Bridges 212 extend between peaks and valleys 210 of the sine wave patterns of adjacent serpentine rings 204. To illustrate, center mesh 106 further includes a second serpentine ring 204B and a third serpentine ring 204C of the plurality of serpentine rings 204. Serpentine ring 204B is connected to serpentine rings 204A, 204C by bridges 212. For example, a second hump 210B of the plurality of humps 210 of serpentine ring 204A is connected to a first hump 210A of the plurality of humps 210 of serpentine ring 204B by a first bridge 212A of the plurality of bridges 212.

In one embodiment, exclusion stent 100A is integral unit, i.e., it is a single piece and not a plurality of separate pieces connected together. For example, exclusion stent 100A is formed by laser cutting a tubular piece of material in the pattern shown for example in FIG. 2.

However, in an alternative embodiment, proximal anchor 102, distal anchor 104, and center mesh 106 start out as separate items (pieces), which are connected together, e.g., by welding. In accordance with this alternate embodiment, the center mesh portion 106 is integral, i.e., serpentine rings 204A-204M are a single piece and not a plurality of separate pieces connected together. For example, serpentine rings 204A-204M are formed by laser cutting a tubular piece of material. Alternatively, serpentine rings 204A-204M are a plurality of separate pieces connected together, e.g., by welding.

In one embodiment, exclusion stent 100A is formed from: 1) stainless-steel; 2) chromium alloy; 3) a shape memory alloy such as nickel titanium that has been alloyed and heat-set, or tempered, in such a manner to provide exclusion stent 100A with an inherent self-expanding characteristic; and/or 4) polymer; and/or 5) a combination thereof, although other materials are used in other embodiments.

To illustrate, proximal anchor 102, distal anchor 104, and center mesh 106 are formed from the same material. Alternatively, proximal anchor 102 and distal anchor 104 are formed from the same material. However, center mesh 106 is formed of a material different than the material of proximal anchor 102 and distal anchor 104.

FIG. 3 is a side plan view of an exclusion stent 100B in accordance with yet another embodiment of the present invention. As shown in FIG. 3, exclusion stent 100B includes proximal anchor 102, distal anchor 104, and a center mesh 106A. Proximal anchor 102 and distal anchor 104 are symmetrically identical to proximal anchor 102 and distal anchor 104 of FIG. 2 and so are not discussed further.

Center mesh 106A is a mesh. More particularly, center mesh 106A is a braided mesh, sometimes called a multiple-juxtaposed mesh or an interlaced mesh.

Proximal anchor 102 is connected to center mesh 106A by bridges 306. More particularly, center mesh 106A includes a proximal, e.g., first, edge 120A and a distal, e.g., second, edge 122A. Bridges 306 extend between peaks 208 of the sine wave pattern of proximal anchor 102 and proximal edge 120A. To illustrate, a first bridge 306A of the plurality of bridges 306 extends between a first peak 208A of the plurality of peaks 208 of the sine wave pattern of proximal anchor 102 and proximal edge 120A. In one embodiment, bridges 306 are formed by welding.

Thus, proximal anchor 102 is connected to proximal edge 120A by bridges 306. Distal anchor 104 is connected to center mesh 106A, and, more particularly, to distal edge 122A of center mesh 106A by bridges 306 in a similar manner and so is not discussed further.

FIG. 4 is a side plan view, in partial cross-section, of exclusion stent 100A of FIG. 2 deployed within a parent artery or vessel 402 of a patient adjacent to a neck 404 of a saccular aneurysm 406 in accordance with one embodiment of the present invention. Illustratively, aneurysm 406 is an intra-cranial aneurysm and parent artery or vessel 402, hereinafter parent vessel 402, is located within the intra-cranial region.

Blood flow through parent vessel 402 is in the direction indicated by arrows 408. Proximal anchor 102 and distal anchor 104 are deployed, sometimes called attached, mounted, wedged, or pressed, against inner vessel wall 410 of parent vessel 402. Proximal anchor 102 and distal anchor 104 are deployed on either side of neck 404, sometimes called an entrance or opening, of aneurysm 406 such that center mesh 106 extends across neck 404 of aneurysm 406.

Since exclusion stent 100A has unit design elements which repeat around the circumference in an endless repeating pattern, deployment of exclusion stent 100A is relatively simple. Exclusion stent 100A is deployed without concern for the rotational orientation of exclusion stent 100A, where the rotational orientation is the angle of exclusion stent 100A around longitudinal axis L. More particularly, the rotational orientation of exclusion stent 100A is not critical since the connection elements of the exclusion stent 100A have a rotationally indistinguishable endless repeating pattern.

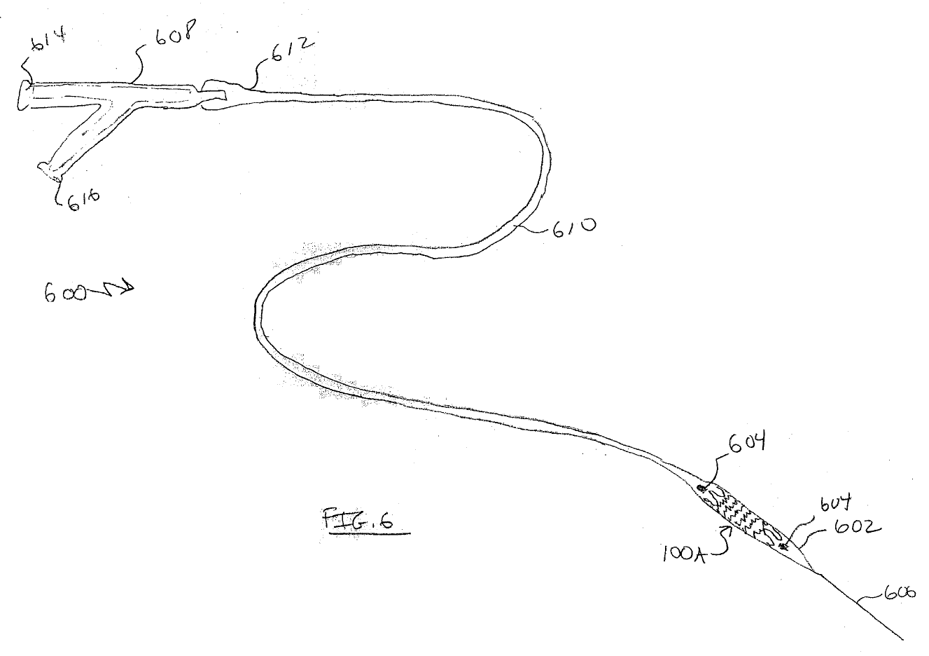

Exclusion stent 100A is simply moved longitudinally within parent vessel 402 until proximal anchor 102 and distal anchor 104 are located on either side of neck 404 of aneurysm 406. Exclusion stent 100A is then deployed such that proximal anchor 102 and distal anchor 104 are deployed against inner vessel wall 410 of parent vessel 402. For example, exclusion stent 100A is deployed using a delivery system configuration 600 of FIG. 6. However, exclusion stent 100A is deployed using other methods well known to those of skill in the art in other embodiments. For example, exclusion stent 100A is deployed using an over the wire, a rapid exchange, or a self-steerable catheter via an integral guide wire technique.

Center mesh 106 extends across neck 404 of aneurysm 406 and thus provides a flow restriction across neck 404. This restriction minimizes and substantially eliminates the pulsatile effect (variations in blood pressure and blood flow due to the heart's pulsing type pumping action) within aneurysm 406 and so that isolation of blood in the aneurysm causes hemostasis within aneurysm 406.

More particularly, center mesh 106 is placed across neck 404 of aneurysm 406, neck 404 being narrow or wide. Center mesh 106 prevents blood flowing through parent vessel 402 from circulating within aneurysm 406 to keep the blood in the aneurysm fluid. The stagnation of blood within aneurysm 406 allows the blood to thrombose, or clot. The thrombus eventually fills aneurysm 406. Once aneurysm 406 is thrombus filled, healing of the parent vessel 402 may reconstruct the inner layer (endothelium) over neck opening 404 thus completely excluding aneurysm 406 from parent vessel 402.

As shown in FIG. 4, exclusion stent 100A is deployed across perforators 412, i.e., small vessels. However, since center mesh 106 only restricts and does not stop blood flow, exclusion stent 100A does not prevent blood from parent vessel 402 from reaching perforators 412. In this manner, complications from occluding perforators 412 are avoided.

As set forth above, aneurysm 406 is a saccular type aneurysm. However, an exclusion stent in accordance with the present invention is also well-suited for use with fistula type aneurysm.

FIG. 5 is a side plan view, in partial cross-section, of exclusion stent 100A of FIG. 2 deployed within parent vessel 402 of a patient adjacent to a fistula aneurysm 506 in accordance with one embodiment of the present invention. Illustratively, aneurysm 506 is an intra-cranial aneurysm and parent vessel 402 is located within the intra-cranial region.

Proximal anchor 102 and distal anchor 104 are deployed against inner vessel wall 410 of parent vessel 402. Proximal anchor 102 and distal anchor 104 are deployed on either side of aneurysm 506 such that center mesh 106 extends across aneurysm 506. The deployment balloon expands the whole stent to a relatively uniform diameter so that there is no bulging or sagging at the location where the stent crosses the aneurysm.

Center mesh 106 extends across aneurysm 506 and thus provides a flow restriction the parent vessel and the sac of the aneurysm 506. This flow restriction minimizes and essentially eliminates the pulsatile effect within aneurysm 506 and causes hemostasis within aneurysm 506 in a manner similar to that described above in reference to FIG. 4.

FIG. 6 is a side plan view of a delivery system configuration 600 for deploying exclusion stent 100A of FIG. 2 in accordance with one embodiment of the present invention. Delivery system configuration 600 includes a catheter 602, which can include an expandable catheter balloon, as those of skill in the art will understand in light of this disclosure. Exclusion stent 100A of FIG. 2 is placed over and mounted on catheter 602. Catheter 602 further includes radiopaque markers 604, which allow the location of catheter 602 to be precisely tracked as the stent is moved to the deployment site over a guidewire (not shown). Catheter 602 further includes a catheter tip 606.

Catheter 602 is connected to a manifold 608 by a catheter shaft 610. In this embodiment, catheter shaft 610 is coupled to manifold 608 by a strain relief portion 612.

Manifold 608 includes a guide wire lumen access port 614 and a balloon inflation and deflation port 616. The guide wire lumen access port 614 guides the catheter 602 over a guidewire to the deployment site through. Further, the catheter balloon of catheter 602 can be pressurized and expanded through balloon inflation and deflation port 616. The guiding of catheters and inflation/deflation of catheter balloons are well known to those of skill in the art.

Referring now to FIGS. 4 and 6 together, exclusion stent 100A is placed over an end portion of catheter 602. Catheter 602, and thus exclusion stent 100A, is introduced intra-vascularly and guided to aneurysm 406. Catheter 602 is moved and manipulated so that exclusion stent 100A is positioned within parent vessel 402 such that proximal anchor 102 and distal anchor 104 are located on either side of aneurysm 406.

Once positioned, the catheter balloon of catheter 602 and exclusion stent 100A are expanded to anchor proximal anchor 102 and distal anchor 104 to inner vessel wall 410 of parent vessel 402. Catheter 602 is then deflated and withdrawn thus leaving exclusion stent 100A permanently implanted within parent vessel 402 as shown in FIG. 4.

However, in an alternative embodiment, exclusion stent 100A is self-expandable. In accordance with this alternative embodiment, exclusion stent 100A, i.e., proximal anchor 102, center mesh 106, and distal anchor 104, self-expands to anchor proximal anchor 102 and distal anchor 104 to inner vessel wall 410 of parent vessel 402 thus avoiding the use of a catheter balloon.

Although exclusions stent 100A is discussed above and illustrated in FIGS. 4, 5, and 6, it is understood that the discussion is equally applicable to exclusion stent 100B of FIG. 3.

FIG. 7 is a side plan view of the region VII of exclusion stent 100A of FIG. 2 in accordance with one embodiment of the present invention. Illustrative specifications for the various characteristics illustrated in FIG. 7 are set forth below in Table 1.

TABLE 1

| CHARACTERISTIC | SPECIFICATION | UNITS |
|---|---|---|
| A7 | 0.0750 | Inches |
| B7 | 0.0065 | Inches |
| C7 | 0.0051 | Inches |
| D7 | 0.0035 | Inches |
| E7 | 0.0395 | Inches |
| F7 | 0.0040 | Inches |
| G7 | 0.0033 | Inches |
| H7 | 0.0038 | Inches |

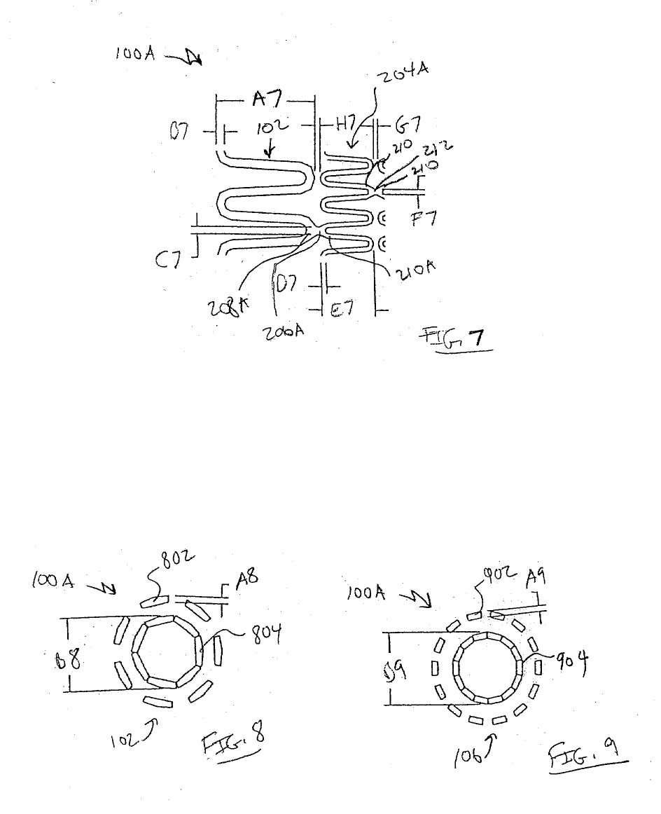

FIG. 8 shows an approximation of two cross-sectional views of proximal anchor 102 along the line VIII-VIII of exclusion stent 100A of FIG. 2 with the expanded state 802 and contracted state 804 both shown aligned with a common center, in accordance with one embodiment of the present invention. Illustrative specifications for the various characteristics illustrated in FIG. 8 are set forth below in Table 2.

TABLE 2

| CHARACTERISTIC | SPECIFICATION | UNITS |
|---|---|---|
| A8 | 0.0045 | Inches |
| B8 | 0.0543 | Inches |

FIG. 9 shows an approximation of two cross-sectional views of center mesh 106 along the line IX-IX of exclusion stent 100A of FIG. 2 with both the expanded state 902 and contracted state 904 shown, both are shown aligned with a common center, in accordance with one embodiment of the present invention. Illustrative specifications for the various characteristics illustrated in FIG. 9 are set forth below in Table 3.

TABLE 3

| CHARACTERISTIC | SPECIFICATION | UNITS |
|---|---|---|
| A9 | 0.0045 | Inches |
| B9 | 0.0538 | Inches |

This disclosure provides exemplary embodiments of the present invention. The scope of the present invention is not limited by these exemplary embodiments. Numerous variations, whether explicitly provided for by the specification or implied by the specification or not, such as variations in structure, dimension, type of material and manufacturing process may be implemented by one of skill in the art in view of this disclosure.

What is claimed is:

1. A structure comprising:
a first anchor; and
a semi permeable cylindrical mesh coupled to said first anchor.

2. The structure of claim 1 wherein said first anchor comprises a pattern.

3. The structure of claim 2 wherein said pattern is a serpentine pattern.

4. The structure of claim 1 wherein said first anchor comprises a serpentine ring.

5. The structure of claim 1 wherein said semi permeable cylindrical mesh comprises a serpentine braid.

6. The structure of claim 1 wherein said semi permeable cylindrical mesh comprises serpentine rings connected together.

7. The structure of claim 6 wherein said first anchor comprises a pattern, said serpentine rings comprising said pattern.

8. The structure of claim 7 wherein said pattern is sinusoidal.

9. The structure of claim 6 further comprising bridges coupling said serpentine rings together.

10. The structure of claim 1 further comprising bridges coupling said first anchor to said semi permeable cylindrical mesh.

11. The structure of claim 1 wherein said semi permeable cylindrical mesh comprises a braided mesh.

12. The structure of claim 11 wherein said semi permeable cylindrical mesh comprises a first edge, said structure further comprising bridges coupling said first anchor to said first edge.

13. The structure of claim 1 further comprising a second anchor coupled to said semi permeable cylindrical mesh, said semi permeable cylindrical mesh extending between said first anchor and said second anchor.

14. The structure of claim 1, wherein said first anchor and said semi permeable cylindrical mesh are self-expandable.

15. The structure of claim 1 wherein said structure is radially symmetric.

16. A structure comprising:
a stent comprising:
a first anchor; and
a semi permeable mesh coupled to said first anchor, said semi permeable mesh comprising a serpentine ring, and
a delivery system configuration comprising a catheter, said stent being on said catheter.

17. The structure of claim 16 wherein said stent is expandable.

18. A structure comprising:
a means for anchoring; and
a semi permeable cylindrical means for restricting a flow of blood coupled to said means for anchoring.

19. The structure of claim 18 further comprising a means for deploying said means for anchoring and said semi permeable cylindrical means for restricting.

20. The structure of claim 18 further comprising a means for coupling said means for anchoring to said semi permeable cylindrical means for restricting.

21. A structure comprising:
a first anchor comprising a sinusoidal pattern;
a semi permeable cylindrical mesh coupled to said first anchor, wherein said semi permeable cylindrical mesh comprises serpentine rings connected together, said serpentine rings comprising said sinusoidal pattern; and
a second anchor coupled to said semi permeable cylindrical mesh, said second anchor comprising said sinusoidal pattern.

22. A method comprising:
placing a stent comprising a first anchor, a second anchor, and a semi permeable cylindrical mesh over a catheter;
guiding said catheter intra-vascularly to an aneurysm of a parent vessel comprising an inner vessel wall;
positioning said first anchor and said second anchor on either side of said aneurysm; and
anchoring said first anchor and said second anchor to said inner vessel wall, wherein said semi permeable cylindrical mesh extends across said aneurysm.

23. The method of claim 22 wherein said anchoring comprises expanding said stent.

24. The method of claim 23 wherein said stent is self-expandable.

25. The method of claim 23 wherein said expanding said stent comprises expanding a catheter balloon of said catheter.

26. The method of claim 22 further comprising restricting a flow of blood through said parent vessel from said aneurysm with said semi permeable cylindrical mesh.
